(19) United States
(12) Patent Application Publication
(10) Pub. No.: US 2022/0068500 A1
(43) Pub. Date: Mar. 3, 2022

(54) SYSTEMS AND METHODS FOR PRIVATELY AND SECURELY TRACKING LOCATION DATA AND BIOMETRIC SAMPLING FOR TRACING DISEASE PROPAGATION

(71) Applicant: Mohammad Abdel-Fattah Abdallah, El Dorado Hills, CA (US)

(72) Inventor: Mohammad Abdel-Fattah Abdallah, El Dorado Hills, CA (US)

(21) Appl. No.: 17/463,545

(22) Filed: Aug. 31, 2021

Related U.S. Application Data
(60) Provisional application No. 63/072,780, filed on Aug. 31, 2020.

(51) Int. Cl. G16H 50/80; G16H 70/60; G16H 10/60; H04L 9/08

(52) U.S. Cl. CPC G16H 50/80; H04L 9/0819; G16H 10/60; G16H 70/60

(57) ABSTRACT

Embodiments of the present invention provide an artificial intelligence-enabled apparatus, such as a mobile communication device equipped with input ports and bio sensors and having a controlling/coordinating/managing methods and software applications running on them for reducing the spread of a pandemic and managing social and economic impact to minimize disruptions that occur due to indiscriminate and global actions such as strict social distancing and business closures.

### CROSS-REFERENCE TO RELATED APPLICATIONS

This application claims the benefit of and priority to provisional patent application Ser. No. 63/072,780, Attorney Docket Number ABDA-P002.PRO, entitled “ARCHITECTING SYSTEMS WITH SMART DEVICES, INTELLIGENT APPARATUS AND TECHNOLOGY METHODS AND/OR AI TRAINED MODELS WITH EDGE/SMART DEVICES AND WEARABLES TO MAINTAIN PRIVACY, ANONYMITY AND CONFIDENTIALITY OF INFORMATION, DATA & TEST SAMPLES” with filing date Aug. 31, 2020, which is hereby incorporated by reference in their entirety as if fully set forth below.

### FIELD

Embodiments of the present invention generally relate to the field of information technology. More specifically, embodiments of the present invention relate to systems and methods for tracking and communicating information securely and confidentially.

### BACKGROUND

Pandemics pose great threat to human life on the health, social, educational and economic levels. One great problem is that individuals in a pandemic might be spreading the illness without others knowing or even they themselves may not be aware of it. This leads to one of two undesired outcomes to manage the problem on the individual level, business/education entities level and government level.

One traditionally used approach is to keep life, social, education and business disruptions at low levels while relying on treating cases at health facilities when they become ill. This approach in a pandemic scenario will lead to massive spread of the illness and inability of the health system to handle the large illness numbers in addition to inability to contain the spread among new individuals. This approach is usually taken because a particular society is unaware of the real underlying early spread of the illness that can go undetected and because no real time efficient mechanism exists to backtrack and trace the increasingly new cases and uncover their potential past infectious history as they were mixing with other individuals.

The other approach is to implement a strict quarantine, stay at home, or even curfew. Such sever measures are indiscriminate and in the long run cause major distribution to life, social, education activities which can cause great harm to the economy.

### SUMMARY

Embodiments of the present invention provide an artificial intelligence-enabled apparatus, such as a mobile communication device equipped with input ports and bio sensors and having a controlling/coordinating/managing methods and software applications running on them for reducing the spread of a pandemic and managing the social and economic impacts to minimize disruptions that occur due to indiscriminate and global actions such as strict social distancing and business closures.

According to one embodiment, a method of securely tracking the location of a device is disclosed. The method includes receiving a plurality of random encrypted private keys at the smart device, where the encrypted private keys correspond to a physical zone, authenticating a physical location of the smart device within the physical zone, selecting a private key corresponding to the physical zone from the random encrypted private keys, sampling a local environmental variable, scrambling the physical location of the smart device using the private key corresponding to the physical zone and a time interval of the sampling, encrypting the physical location of the smart device using the environmental variable, and transmitting the physical location of the smart device as encrypted to a remote computer system.

According to some embodiments, the method includes determining that the smart device is associated with a potential infection and transmitting the physical location of the smart device to a plurality of smart devices.

According to some embodiments, the method includes determining that at least one of the plurality of smart devices is physically located near the physical location of the smart device, and transmitting a message to the at least one of the plurality of smart devices physically located near the physical location of the smart device, where the message indicates a potential exposure.

According to some embodiments, the method includes receiving new encrypted private keys at regular intervals.

According to some embodiments, the location within the physical zone includes x dimension data and y dimension data.

According to some embodiments, authenticating that the smart device is physically located within the physical zone includes performing triangulation.

According to some embodiments, the method includes identifying a second smart device that is physically located within the physical zone at the same time as the smart device.

According to some embodiments, the local environmental variable includes at least one of ambient temperature, atmospheric pressure, and humidity.

According to some embodiments, the local environmental variable includes a communication signal strength between the smart device and a communication tower.

According to some embodiments, the transmitting the location within the physical zone as encrypted to a remote computer system includes transmitting the location within the physical zone as encrypted over a zero log VPN channel.

According to a different embodiment, a system for securely tracking the location of a device is disclosed. The system includes an apparatus that broadcasts encrypted private keys, where the encrypted private keys correspond to a respective physical zone, and a smart device in communication with the apparatus and executing a software application that performs a method including authenticating that the smart device is physically located within a physical zone, selecting a private key corresponding to the physical zone from the encrypted private keys broadcast by the apparatus, sampling a local environmental variable, scrambling a physical location of the smart device using the private key corresponding to the physical zone and a time interval of the sampling, encrypting the location within the physical zone using the environmental variable, and transmitting the physical location of the smart device as encrypted to the apparatus.

According to some embodiments, the system further includes a plurality of second smart devices, and where the apparatus determines that the smart device is associated with a potential infection, and transmits the physical location of the smart device to the plurality of second smart devices.

According to some embodiments, the apparatus determines that at least one of the plurality of second smart devices is physically located near the physical location of the smart device and transmits a message to the at least one of the plurality of second smart devices physically located near the physical location of the smart device, where the message indicates a potential exposure.

According to some embodiments, the method includes receiving new encrypted private keys at regular intervals.

According to a different embodiment, the secure biometric sampling apparatus is disclosed. The apparatus includes a finger scanner configured to measure oxygen levels and pulse rate, a micro needle device, a processor in electronic communication with the finger scanner and the micro needle device that executes instructions to automatically perform a method including receiving biometric data from the finger scanner and the micro needle device, authenticating a user according to the biometric data, and determining that the user is associated with a potential infection according to the biometric data.

According to some embodiments, the method further includes receiving second biometric data from the finger scanner and the micro needle, where the determining that the user is associated with a potential infection according to the biometric data includes comparing the first biometric data to the second biometric data.

According to some embodiments, the method further includes training a machine learning application using the biometric data.

According to some embodiments, the method further includes extracting biomarker features from the biometric data.

According to some embodiments, the determining that the user is associated with a potential infection is further performed according to the biomarker features.

According to some embodiments, the method further includes transforming the biometric features to a residue number system.

### BRIEF DESCRIPTION OF THE DRAWINGS

The accompanying drawings, which are incorporated in and form a part of this specification, illustrate embodiments of the invention and, together with the description, serve to explain the principles of the invention:

FIG. 1A depicts an exemplary intelligent apparatus operating in multiple zones to securely trace infection propagation according to embodiments of the present invention.

FIG. 1B depicts an exemplary sequence of computer implemented steps for automatically authenticating devices using a key associated with a zone in combination with local environment variables according to embodiments of the present invention.

FIG. 2 depicts an exemplary sequence of computer implemented steps for extracting samples of biometric features for AI training while preventing the samples from being linked or associated with features to the original according to embodiments of the present invention.

FIG. 3 depicts an exemplary test device that registers the fingerprint of a finger inserted into the device using a fingerprint scan area while also measuring oxygen level and pulse rate according to embodiments of the present invention.

### DETAILED DESCRIPTION

Reference will now be made in detail to several embodiments. While the subject matter will be described in conjunction with the alternative embodiments, it will be understood that they are not intended to limit the claimed subject matter to these embodiments. On the contrary, the claimed subject matter is intended to cover alternative, modifications, and equivalents, which may be included within the spirit and scope of the claimed subject matter as defined by the appended claims.

Furthermore, in the following detailed description, numerous specific details are set forth in order to provide a thorough understanding of the claimed subject matter. However, it will be recognized by one skilled in the art that embodiments may be practiced without these specific details or with equivalents thereof. In other instances, well-known methods, procedures, components, and circuits have not been described in detail as not to unnecessarily obscure aspects and features of the subject matter.

Reference will now be made in detail to several embodiments. While the subject matter will be described in conjunction with the alternative embodiments, it will be understood that they are not intended to limit the claimed subject matter to these embodiments. On the contrary, the claimed subject matter is intended to cover alternative, modifications, and equivalents, which may be included within the spirit and scope of the claimed subject matter as defined by the appended claims.

Furthermore, in the following detailed description, numerous specific details are set forth in order to provide a thorough understanding of the claimed subject matter. However, it will be recognized by one skilled in the art that embodiments may be practiced without these specific details or with equivalents thereof. In other instances, well-known methods, procedures, components, and circuits have not been described in detail as not to unnecessarily obscure aspects and features of the subject matter.

Portions of the detailed description that follows are presented and discussed in terms of a method. Although steps and sequencing thereof are disclosed in a figure herein describing the operations of this method, such steps and sequencing are exemplary. Embodiments are well suited to performing various other steps or variations of the steps recited in the flowchart of the figure herein, and in a sequence other than that depicted and described herein.

Some portions of the detailed description are presented in terms of procedures, steps, logic blocks, processing, and other symbolic representations of operations on data bits that can be performed on computer memory. These descriptions and representations are the means used by those skilled in the data processing arts to most effectively convey the substance of their work to others skilled in the art. A procedure, computer-executed step, logic block, process, etc., is here, and generally, conceived to be a self-consistent sequence of steps or instructions leading to a desired result. The steps are those requiring physical manipulations of physical quantities. Usually, though not necessarily, these quantities take the form of electrical or magnetic signals capable of being stored, transferred, combined, compared, and otherwise manipulated in a computer system. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussions, it is appreciated that throughout, discussions utilizing terms such as “accessing,” “writing,” “including,” “storing,” “transmitting,” “associating,” “identifying,” “encoding,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

Some embodiments may be described in the general context of computer-executable instructions, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

### Systems and Methods for Privately and Securely Tracking Location Data and Biometric Sampling for Tracing Disease Propagation

Embodiments of the present invention provide an artificial intelligence-enabled apparatus, such as a mobile communication device equipped with input ports and bio sensors and having a controlling/coordinating/managing methods and software applications running on them for reducing the spread of a pandemic and managing social and economic impact to minimize disruptions that occur due to indiscriminate and global actions such as strict social distancing and business closures.

Some embodiments describe a complete system that can be created by embodying individual personal electronic devices (e.g., wearables or mobile communication devices, mobile smart phones, smart wearables) with additional hardware sensing components that combine the use of device input ports e.g., image/video capture, audio capture, touch capture, etc.) based on uniquely identifiable individual feature sensors, biometrics sensors, and a logically central (and optionally physically distributed) apparatus. AI and machine learning hardware and algorithms can be used to perform training and analysis/prediction of illness/infectious status employing a managing method and application to form a new system using new methods to train and predict infectious status and use those as parameters to help facilitate actions such as entry passes, boarding a mass transit, class attendance permit or entry through country border. Embodiments can also enable the private recording of history of space and time records that are facilitated using the central (or distributed) apparatus to identify, track and trace potentially infected/infectious individuals who were collocated (in timeframe & space) in the past.

The tracking and tracing of past colocation history can be performed when the infectious status of an individual is known to be unsafe or predicted by the AI apparatus as such later in time as all the records can identify the people who were collocated with the individual in the past. This will be beneficial if the devices and the managing application are implemented on a wide-spread basis, such as enhancing a mobile smart phone with those capabilities, and combining additional bio sensors with new/modified uses of existing traditional sensors (e.g., image, video, audio, touch) and implementing and constructing new methods through a managing system connected to an AI/ML central prediction, recording and tracking/tracing apparatus (e.g., hardware & software server infrastructure of distributed processing elements (e.g., server farm, computing cloud, etc.)).

Using these novel approaches to trigger the tracing and tracking as opposed to traditional ways can achieve much more large scale encapsulation of potential positive cases than testing alone and it can also achieves that faster than testing. Not everyone who is infected will reach the state where they will seek medical evaluation or performing a test, because many cases can be mild or with light symptoms, moreover the AI based prediction can reach potential diagnosis faster and more seamlessly than physically reaching the point of seeking medical help and testing. Such factors can make a huge difference on the infection curve to significantly reduce further spread by using the AI triggered tracing and tracking.

The individual participating with their device in this wide scale system will be the one who is notified with their predicted infectious status, also he is the one who will be notified of being collocated in the past and with another individual who later got a confirmed or predicted infectious status. Also the individual himself based on his own predicted status which his device obtained from the central apparatus (or in another embodiment performing part or all of the inference/prediction on his device) privately will enable him to obtain safe passes for entering events or participate in gatherings or mass transit or crossing borders. If he has a potential non-safe status, then he needs to do self-quarantine (which can be potentially verified by the device location history during the quarantine time and proximity detection of other individuals) or obtain formal medical testing or evaluation clearness. If he did either of those actions, then his status will change on his device to potentially safe status.

All predicted safe/unsafe status is not a guarantee of such status, a major purpose of the system is to mitigate and reduce but cannot eliminate or guarantee any safe or unsafe status as there are infected individuals with completely undetected symptoms who even stay unidentified even with the use of AI systems, so other preventive and protective measures such using masks, cleaning and washing hands and not touching face, mouth, nose, etc., may still be required, as well as maintaining enough distance from each other in any colocation situation.

In situations where privacy and confidentiality is crucial, it is important for any technology or problem solutions to take that into consideration, for example addressing health information such as infectious status, disease and medical conditions as well as personal information such as location history, interpersonal presence, and communication records. In many cases, that same information is needed to enable important aspects of a particular solution such as the example application mentioned earlier. This creates a technological challenge of how to use that private information while at the same time keeps confidentiality. In the current invention multiple innovative solutions will be discussed, at the same time variations of the applications and/or the embodiments is possible within the context of this invention.

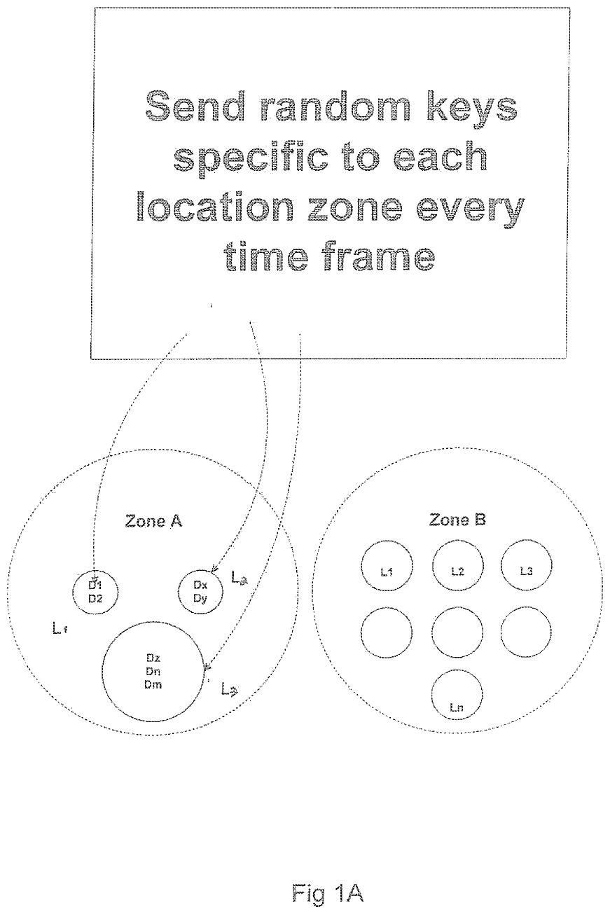

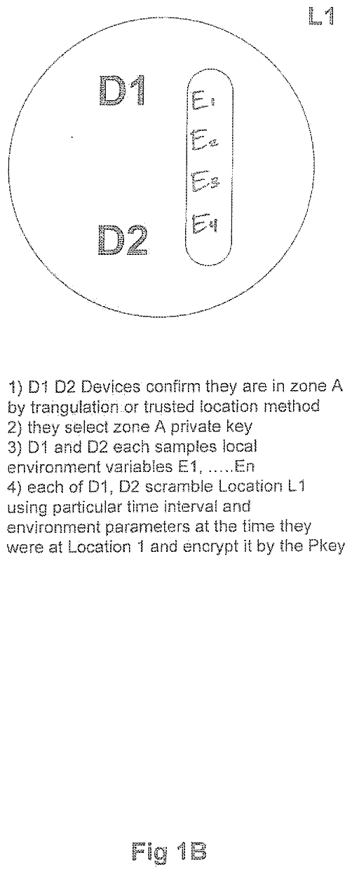

With regard to FIG. 1A, an exemplary intelligent apparatus operating in multiple zones is depicted according to embodiments of the present invention. The intelligent apparatus operates in zone A and zone B to keep private information concealed while being able to broadcast information and use it for comparison purposes to detect infection, for example. A contact tracing application is implemented using the intelligent apparatus to detect the presence of a person who is contagious in a particular location at some point in time, and the application can trace other people who either came in close proximity to locations that person had visited within a certain time frame (not necessarily at the same time). At every time frame of the application, the intelligent apparatus sends random keys specific to each location zone (e.g., Zone A or Zone B). Zone A includes devices D₁ and D₂ at location L₁, devices Dₓ and D_{y} located at location L₂, and devices D_{z}, Dₙ, and Dₘ located at L₃. Zone B can include several different locations and devices as well, which are different than the locations and devices in Zone A.

Smart devices and intelligent apparatus are used to enable tracking and tracing of infection propagation without the revealing of identity of either the person with infection or the person at risk or information about their actual location or time of colocation. In the example of a pandemic, it is important to identify colocation overlap in time inside indoor environments, while for outdoor cases, Bluetooth proximity schemes are sufficient to detect possible coinfection. The smart device hosts an application paired with the intelligent apparatus, the intelligent apparatus broadcasts random encrypted private keys to the application on smart devices. Those keys are different for different location zones (a location zone is preset areas of x and y dimensions), they also change every timeframe period, the application running on the smart device in every time frame identify the location zone it is located in using a trusted location identification method, such as direct/secured GPS access or signal triangulation method of nearby communication towers. This is done to prevent other devices from using a fake location service to trick the application to think it is in a particular zone.

After the corresponding key is identified by the user device, it is used to encrypt a more fine-tuned location (e.g., L₁) within the zone. Before encrypting the fine tuned location it is augmented (scrambled) by a more fine tuned interval within the time frame. Furthermore it is augmented/scrambled by local environment parameters measured by the smart device and then hashed by one-way hash function (like using SHA), those local environment parameters are not easily accessible by an external entity, such local parameters could be in one embodiment using the indoor ambient temperature, measured by the smart device sensors. It could also be the atmospheric pressure at the particular elevation of the indoor floor the smart device is currently located at, also it is possible to use indoor humidity, as well as communication signal strength from nearby towers within indoors. It is important to note that it is not necessary to use exact values for those environment parameters, but a bounding range can be used instead (rounded to certain digit/bit). It is possible also to limit the location storage of encrypted locations to locations where the device was indoors.

The final augmented (scrambled) and one-way hashed location is then encrypted using the particular zone/timeframe broadcasted private key. Then the encrypted augmented/scrambled and hashed location is stored securely on the device. This can happen every time-interval within each timeframe.

Once the intelligent apparatus predicts or confirms an infection of a smart device user, user device sends its encrypted augmented/scrambled and hashed locations history to the intelligent apparatus using for instance a zero log VPN channel. The user device regularly sends information to the smart apparatus for AI training, and in some embodiments, for inference and other type of information. Once the intelligent apparatus receives the encrypted message with hidden content of confirming/predicting past infection, it then broadcast those encrypted scrambled locations to all user smart devices, which on their part compares it to their own stored encrypted scrambled & hashed locations.

If a match is found then the device user is at risk of being collocated with an infected person in the past. This way actual locations of users never leave their devices and even when they are predicted to be infectious, the encrypted augmented/scrambled locations that are sent out are difficult to be compared to a specific location by a monitoring/observer agent, first they are scrambled using time variant environmental local parameters mentioned earlier. This makes it very difficult for a remote observer/agent to try to fake its location (as the smart device installed application will use multiple methods to confirm approximate location to minimize the chances of such location faking), but also because the application will use local environmental time variant parameters to scramble the location using one way hashing function and encrypting it. So if a remote observer tries to monitor a specific location to find out the time frame that any user who become particularly infected has visited that location to try to infer such user, he will not be able to do that. The always time changing encryption key for each zone will also make it difficult to keep track of all those variables.

In another embodiment, the intelligent apparatus generates a list of large prime numbers to be bases for the encryption specific for each location zone and time frame (that also keep changing every time) based on the location zone and timeframe. The smart user devices collocated in a particular timeframe and location can use the fine-tuned time interval (within the timeframe) alongside local environment parameters/variables (as mentioned previously) to select a subset of the large prime numbers used as bases for the encryption. For example, if the set of large prime numbers sent by the intelligent apparatus to a particular zone at a particular time frame include 40 such large prime numbers, each of the user smart device then chooses 2 of this list based on the local environment parameters at the location and within that time interval.

In another embodiment it is possible to only use cryptographic one-way hash functions (such as SHA) without using the top layer additional encryption or vice versa (e.g., using the encryption top layer without using a one-way hash function for the location but rather use only scrambling technique based on the local environment parameters alongside the finetuned time interval). If only cryptographic hash function layer is used with local environment variables and time interval then user smart device can choose a version of cryptographic one way hash function (or change the parameters of the function) from a list sent by the intelligent apparatus for that particular zone and time frame by using the finetuned time interval and local environment parameters as basis for the selection as well as use them to scramble the location.

It is worth noting that some embodiments can pick and choose different combinations of the methods used above using different hardware and software implementations and similarly such implementations can use those described methods with the combination of systems of hardware and software to be applied to establish an anonymous link (that link being kept confidential except for the parties involved) to things other than just location, including ability to anonymously link physical or virtual objects that share similarities because they were doing something similar (physically or virtually). As an example, persons or computers virtually visit a website within a certain timeframe, another example is the ability to anonymously link a person to other people who studied in a particular online university or who shared a particular hobby experience.

In one example, an intelligent apparatus and user smart devices anonymously stores past experiences such as reading novels or searching online for particular items to purchase or evaluate. An intelligent apparatus role as described above sends out the list of encryption keys or one way hash functions based on generic characteristics such as novel reading, sport playing or furniture shopping or rating/ranking/review of particular item categories or experiences (e.g., timeframes of such activities or experiences). The smart devices use those keys/function lists sent by the intelligent apparatus alongside other localized/personalized environment variables (e.g., time intervals) such as the titles of the novels they read or the authors of those novels, or some particular characteristics of the items they were searching for or wanted to purchase such as price range, manufacture, material of the item, sharing of a particular experience etc. Such activity specifics/characteristics can be stored on the device. Although it is possible to input localized or personalized local parameters manually if it is through trusted medium, it is much more scalable and useful to do that seamlessly. In the examples above for example when reading a novel online, the smart device obtain the encryption key sent by the intelligent apparatus related to accessing the novel section of the online reading website, while the local environment variable might include any special interest category such as genre of the novel also obtained from the website when the novel is actually being read from the online website or when it actually got purchased electronically. Other more specific localized/personalized environment parameters can be things like time interval it took this particular user to finish reading the novel.

Other example of the localized/personalized characteristics or experiences is while shopping for certain items online, if the items purchased were on promotion when bought, other examples will be for travel experiences, the intelligent apparatus can share keys and hash function lists related to a particular hotel chain or particular tourist point of interest. While the local variables might be the number of pictures or selfies taken or the intensity of the physical activities captured by the smart user device at that spot. The users who shared the similar experiences or activities, can be anonymously linked together to share or inquire more information about those experiences and activities without revealing identity at all or even publicly disclose information with others except those who had similar shared experiences. Those with similar shared experiences can be anonymously linked together and be able to extract some additional information about those shared experiences and even be able to anonymously be linked together upon mutual consent. This can prevent others who did not share those experiences from acquiring any information about those experiences or be able to link with those who actually had those experiences. This sharing of the information among those who shared the experiences is something that is permitted by the device user to start with. This has the potential to create platform of anonymous direct user interaction and information linking and sha ring without publicly being available for others who did not actually share the activity or the experience.

FIG. 1B depicts an exemplary sequence of computer implemented steps for automatically authenticating devices using a key associated with a zone in combination with local environment variables according to embodiments of the present invention. In FIG. 1B, Zone A and includes devices D₁ and D₂ and environmental variables or conditions E₁, E₂, E₃, . . . , Eₙ at location L₁.

At step 1, devices D₁ and D₂ confirm they are physically located in Zone A using triangulation or other trusted location method.

At step 2, devices D₁ and D₂ select the Zone A private key.

At step 3, devices D₁ and D₂ each sample at least one local environment variable E₁, E₂, E₃, . . . , Eₙ.

At step 4, each device D1 and D2 scramble location L₁ using a specific time interval and environment parameter at the time they were physically present at location L_{1 and} perform encryption using the Zone A private key.

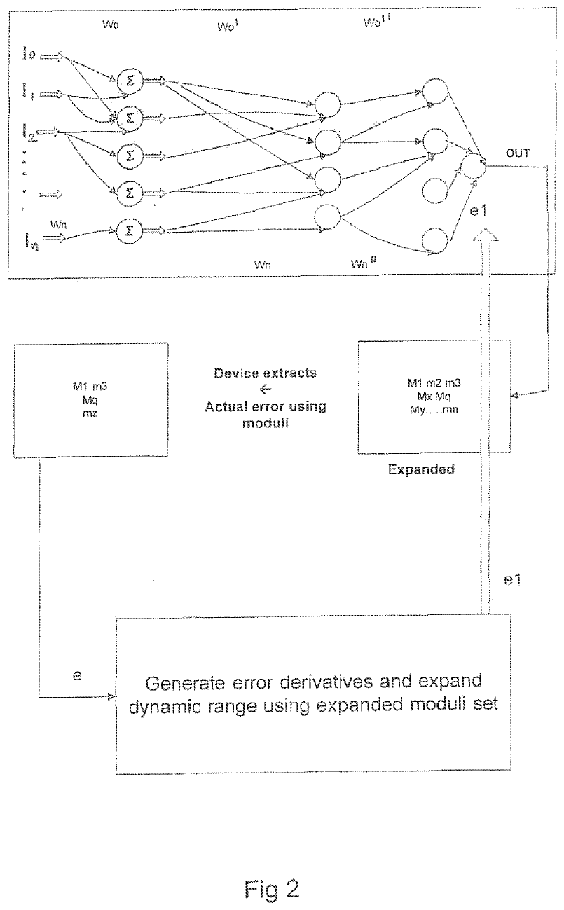

FIG. 2 depicts an exemplary sequence of computer implemented steps of a method for extracting samples of biometric features for AI training while preventing the samples from being linked or associated with features to the original according to embodiments of the present invention. In one example image or voice samples of a person are used to monitor any changes that can indicate a change in the health or aid in AI medical diagnosis.

According to some embodiments, samples of voice, image and touch of device users and authenticated by user biometrics captured simultaneously alongside the samples extracted biomarkers are protected using biomarkers that train an AI machine learning intelligent apparatus. One way to address that is to divide the input sample into blocks that represent only portions of the sample and distribute those blocks on distributed machine learning apparatuses, the drawback is some observer intercepting/gathering the distributed blocks to obtain the original sample. Another way is to encrypt or one-way hash the sample data and use the encrypted or one-way hashed samples for training and prediction. It is difficult to assume that this will not impact the model accuracy and performance. The other way is to obfuscate the sample data with noise that still approaches good prediction accuracy.

Some embodiments use features extracted from sample data transformed into RNS (residue number system) remainders using a group of selected moduli by the user device, the sensitive sample data after being transformed into multiple RNS digits/remainders get augmented by other RNS imprecise digits of expanded module sets (only the user smart device knows which are the actual module sets that represent the actual data and which are the expanded/ im precise module set digits).

In one embodiment the user device captures samples of something like the voice or image those first are authenticated with biometrics to ensure that the biomarkers features extracted later in the process are simultaneously linked to the user through this biometric authentication. Then the voice samples are scanned locally to extract the important biomarkers features (this could also include using basic artificial intelligence/deep learning algorithm locally on the device) this is used to create snippets of biomarkers of significance and extract their features (as in catching breath between words or snippets of long vowels, etc. It also includes creating local micro portions of the eye or face images and extracting features of snippets of biomarkers of significance from those images).

It is important to note that the process is localizing the biometric authentication of images, voice and touch to the user device and also the extracted features of biomarkers of significance that will be sent out to the intelligent apparatus. This is the first stage to minimize exposure of plain sample data. The next stage is hiding the extracted features values in an expanded numerical domain using the expanded RNS domain. Most deep learning networks rely on computation using Matrix math (subsequent multiply accumulate calculations) and use of rectifying functions, both in this invention are suggested to be carried using RNS arithmetic in an orthogonal way among the RNS digits within each modulus.

The extracted biomarker features of significance numerical values is transformed into the RNS digits in each RNS module channel (the remainders of dividing the sample value by each of the base set chosen moduli) then the sample values is modified using a distortion function (which does not completely distort the sample extracted features values but shift it, as in adding some noise to it and expanding its dynamic range by an additional set of moduli thus forming an expanded moduli set). Then the total resulting RNS digits of the expanded set is send to the intelligence apparatus for processing which can be mostly carried out in each RNS modulus channel separately at least in the forward propagation pass of the neural network training phase; the resulting outcome of the forward phase is sent to the user device as the digits of all RNS modulus including the base set and the expanded set moduli. It is only the user device that have the original extracted feature value and knows the actual base set and the extra moduli for the expanded set. The user smart device calculates the adjusted output value using the actual moduli set and compensate for the expanded set if needed. The error between the adjusted value and the extracted feature reference value on the device is then calculated and sent back to the intelligent apparatus for the backward propagation phase of the network training but again using the expanded set of moduli and adjusting with some noise on part of the error in the expanded moduli set. This way the error in the forward pass is corrected using actual outcome calculated on the user device but the intelligent apparatus does not know the actual values.

The expanded set is selected to be larger than the base set to increase the space of potential input features values, if the base set has x moduli and the expanded set has 2×, then the exploration space for an observer agent to find the real moduli set is x chosen out of 2×, for example x=20 then this has billions of possible outcomes to choose the basic moduli set from. This is hard to guess especially that the features themselves are extracted from the original samples through a stage of eliminating identifiable biometrics features and keeping only features of biomarkers of significance and adjusting with noise as explained earlier. There is not a straight forward way to take the RNS expanded set representation of the extracted noise-adjusted features (done on the user device through the first stage) and then find a way to correlate 900 billion possible outcomes (trying to guess the actual RNS base set) with yet unknown reference for the comparison (such as extracted features of biomarkers of significance from the images or sounds or photos of a large population). It is important to note that although AI systems using RNS expanded and base moduli set can be used for both training and inference (prediction), it is possible in preferred embodiment to only perform the training of the AI model on the intelligent apparatus using the expanded moduli set to compute the error and then refine that error and its backpropagated derivatives on the smart devices using the actual moduli set and then backpropagate it through the trained AI model on the intelligent apparatus by expanding the dynamic range to the expanded moduli set, while at the same time the inference (prediction) is performed on the smart device using the shrink AI model with only the base set of moduli on the smart device. This can be done by importing the weights from the trained model and only using the model in its shrink form with only base moduli set weight components/digits and calculations on the user device.

It is important also to note that the method, apparatus and embodiment of using the RNS scheme illustrated above can also be used to solve the location privacy mentioned above in a different way, that can be achieved by using the local parameters (obtained from the local user smart device environment) to choose the expanded set of moduli, whereas the base set of moduli is sent encrypted to the user device whereas different base set moduli are sent for each user device native location zone and particular time frame. The user device then uses local environment parameters to select additional sets of moduli to create the expanded set in which the encrypted/one way hashed and adjusted values of location and time frame can be represented with. Later when the encrypted and range extended RNS numerical representation are broadcasted to other devices in case of history location contamination, then each user device compare their actual RNS base set & encrypted values with the broadcasted RNS expanded moduli set after ignoring the expanded moduli (as they would have stored and identified the expanded moduli associated with each timeframe in their history) and thus they can compare by excluding those moduli in the expanded set and use the base moduli set for comparison.

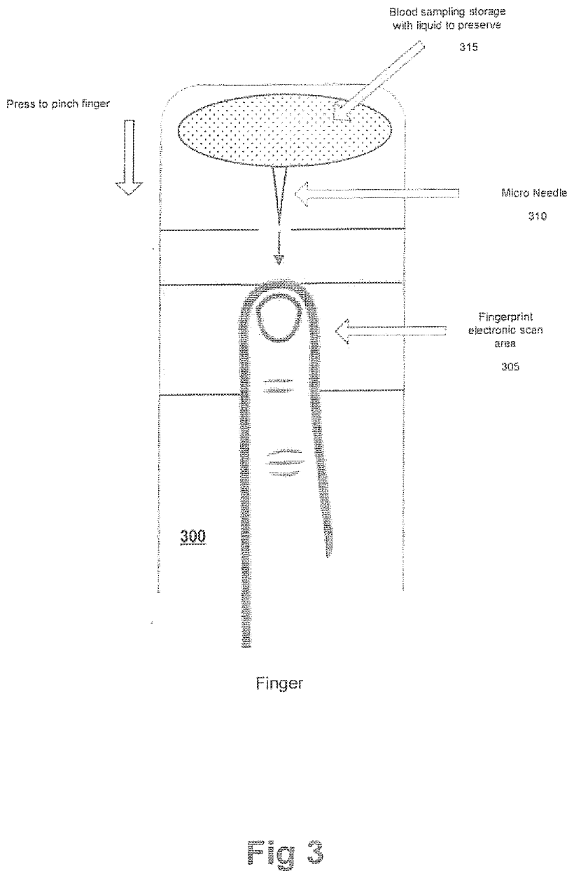

FIG. 3 depicts an exemplary bio-authenticated instrument that extracts biomarkers authenticated to the user for use by virtual health and telemedicine according to embodiments of the present invention. The bio-authenticated instrument can perform biometric authentication using face, voice or fingerprint recognition to link the biomarkers and bio data to the user without revealing the identity.

According to some embodiments, biometric authentication is performed to enable remote and virtual healthcare, where remote testing can be carried out using an intelligent apparatus by linking the bio data results of the tests or the measurements. The test or measurements can be carried our remotely, for example, by simultaneously linking diagnosis based on face/eye image features (color, dehydration, slimness) and/or face recognition, voice/sound features (hoarseness, breath patterns, cough, inflammation based vibrations in vocal cords) and using light through/reflection or reflected by skin to measure oxygen level, temperature measurement through finger touch, or fingerprint image. These simultaneous dual action of diagnoses and authentication linkage help establish trust and privacy for those applications for remote and virtual health, some of those methods and embodiments can be carried out with repurposing smart devices and wearables but moreover other devices can be created on this same innovative principle.

In the embodiment depicted in FIG. 3, the test device 300 to registers the fingerprint of a finger inserted into the device using fingerprint scan area 305 while also measuring oxygen level and pulse rate through light absorption measurement (e.g., an oximeter), the outcome is a bio authenticated and bio linked measurement that can be trusted for remote or virtual health.

Test device 300 includes a micro needle device 310 that pinches the fingertip and absorbs small amount of blood 315 that be analyzed within the micro needle device (e.g., using a glucose meter), or the blood sample 315 can be automatically sealed/conserved in a small container that can be used as part of home test kits. The test device 300 can include a smart device/wearable with a camera having infrared capability can be used to measure user face temperature while simultaneously authenticate the user bio identity and link it to the user biomarker (in this case his temperature), in a similar way sound biomarkers (like cough or voice hoarseness or breath catch patterns) can be use with voice biometric recognition this biometric authenticated biomarkers/bio data using smart devices can be shared for remote or virtual health applications. The trusted environment created by such smart devices and authenticated mechanisms can foster a reliable remote/virtual health system. The test device 300 can also include a microphone for sampling voice data, for example.

Embodiments of the present invention are thus described. While the present invention has been described in particular embodiments, it should be appreciated that the present invention should not be construed as limited by such embodiments, but rather construed according to the following claims.

What is claimed is:

1. A method of securely tracking a location of a first smart device, the method comprising:
receiving a plurality of encrypted private keys at the first smart device, wherein the encrypted private keys correspond to a physical zone;
authenticating a physical location of the first smart device within the physical zone;
selecting a private key corresponding to the physical zone from the plurality of encrypted private keys;
sampling a local environmental variable;
scrambling the physical location of the first smart device using the private key corresponding to the physical zone and a time interval of the sampling;
encrypting the physical location of the first smart device using the environmental variable; and
transmitting the physical location of the first smart device as encrypted to a remote computer system.

2. The method as described in claim 1, further comprising:
determining that the first smart device is associated with a potential infection; and
transmitting the physical location of the first smart device to a plurality of smart devices.

3. The method as described in claim 2, further comprising:
determining that at least one of the plurality of smart devices is physically located near the physical location of the first smart device; and
transmitting a message to the at least one of the plurality of smart devices physically located near the physical location of the first smart device, wherein the message indicates a potential exposure.

4. The method as described in claim 1, further comprising receiving new encrypted private keys at regular intervals.

5. The method as described in claim 1, wherein the location within the physical zone comprises: x dimension data; and y dimension data.

6. The method as described in claim 1, wherein authenticating that the first smart device is physically located within the physical zone comprises performing triangulation.

7. The method as described in claim 1, further comprising identifying a second smart device that is physically located within the physical zone at the same time as the first smart device.

8. The method as described in claim 1, wherein the local environmental variable comprises at least one of: ambient temperature; atmospheric pressure; and humidity.

9. The method as described in claim 1, wherein the local environmental variable comprises a communication signal strength between the first smart device and a communication tower.

10. The method as described in claim 1, wherein the transmitting the location within the physical zone as encrypted to a remote computer system comprises transmitting the location within the physical zone as encrypted over a zero log VPN channel.

11. A system for securely tracking a location of a first smart device, the system comprising:
an apparatus operable to broadcast encrypted private keys, wherein the encrypted private keys correspond to a respective physical zone; and
the smart device operable to engage in communication with the apparatus and further operable to execute a software application that performs a method comprising:
authenticating that the first smart device is physically located within a physical zone;
selecting a private key corresponding to the physical zone from the encrypted private keys broadcast by the apparatus;
sampling a local environmental variable;
scrambling a physical location of the first smart device using the private key corresponding to the physical zone and a time interval of the sampling;
encrypting the location within the physical zone using the environmental variable; and
transmitting the physical location of the first smart device as encrypted to the apparatus.

12. The system as described in claim 11, wherein the apparatus is operable to determine that the first smart device is associated with a potential infection, and further operable to transmit the physical location of the first smart device to a plurality of smart devices.

13. The system as described in claim 12, wherein the apparatus is further operable to determine that at least one of the plurality of smart devices is physically located near the physical location of first the smart device and further operable to transmit a message to the at least one of the plurality of smart devices physically located near the physical location of the first smart device, wherein the message indicates a potential exposure.

14. The system as described in claim 11, further comprising receiving new encrypted private keys at regular intervals.

15. A secure biometric sampling apparatus, the apparatus comprising:
a finger scanner configured to measure oxygen levels and pulse rate;
a micro needle device;
a processor in electronic communication with the finger scanner and the micro needle device, wherein the processor is operable to execute instructions to automatically perform a method comprising:
receiving biometric data from the finger scanner and the micro needle device;
authenticating a user according to the biometric data; and
determining that the user is associated with a potential infection according to the biometric data.

16. The secure biometric sampling apparatus of claim 15, wherein the method further comprises receiving second biometric data from the finger scanner and the micro needle, wherein the determining that the user is associated with a potential infection according to the biometric data comprises comparing the first biometric data to the second biometric data.

17. The secure biometric sampling apparatus of claim 15, wherein the method further comprises training a machine learning application using the biometric data.

18. The secure biometric sampling apparatus of claim 15, wherein the method further comprises extracting biomarker features from the biometric data.

19. The secure biometric sampling apparatus of claim 18, wherein the determining that the user is associated with a potential infection is further performed according to the biomarker features.

20. The secure biometric sampling apparatus of claim 18, wherein the method further comprises transforming the biometric features to a residue number system.
